(19) United States
(12) Patent Application Publication
(10) Pub. No.: US 2025/0248590 A1
(43) Pub. Date: Aug. 7, 2025

(54) ENDOSCOPE VALVE DEVICES, SYSTEMS, AND METHODS

(71) Applicant: BOSTON SCIENTIFIC SCIMED, INC., MAPLE GROVE, MN (US)

(72) Inventors: Nathan Thomas Cummings, Worcester, MA (US); Julia Veitch, Waltham, MA (US); Brian Luis, Boylston, MA (US)

(73) Assignee: BOSTON SCIENTIFIC SCIMED, INC., MAPLE GROVE, MN (US)

(21) Appl. No.: 18/429,633

(22) Filed: Feb. 1, 2024

(51) Int. Cl. A61B 1/015; A61B 1/00

(52) U.S. Cl. CPC A61B 1/015; A61B 1/00068

(57) ABSTRACT

A valve assembly with a valve shaft shiftable within a valve well from an off position blocking fluid communication between ports in the valve well, and an on position allowing fluid communication between such ports. The valve shaft is in a stable position in either the off position or the on position such that force need not be applied to maintain the valve shaft in either the off position or the on position once the valve shaft has been moved to such position.

### CROSS-REFERENCE TO RELATED APPLICATIONS

This application claims the benefit of priority under 35 U.S.C. § 119 to U.S. Provisional Application No. 63/442,789, filed Feb. 2, 2023, the entire disclosure of which is hereby incorporated by reference herein for all purposes.

### FIELD

The present disclosure relates generally to devices (including, without limitation, components and assemblies), systems, and methods for controlling flow of materials through a valve. In particular, the present disclosure relates to devices, systems, and methods for controlling flow of materials through a valve assembly usable in a medical device such as an endoscope.

### BACKGROUND

Various devices with valve assemblies are known in the art for use during various medical procedures to control fluid flows. For instance, materials may be supplied to an anatomical site (e.g., fluid may be supplied, such as for irrigation), and/or suctioned from the anatomical site (e.g., fluid or biological materials may be withdrawn from an anatomical site) during a medical procedure. A valve assembly may be used to control flow of such materials. An endoscope is a common medical device used to introduce or remove substances with respect to an anatomical site, and thus typically includes a valve assembly. Endoscopes typically have an insertion tube with a working channel via which substances (e.g., fluids such as gas or liquids) or devices or instruments or tools may be introduced to an anatomical site, or substances may be removed or suctioned out from an anatomical site. A valve assembly is typically associated with a control handle of the endoscope and in fluid communication between a fluid supply and/or vacuum source and the endoscope's insertion tube to control flow of substances through the endoscope. The valve assembly typically has a valve well and a valve shaft shiftable within the valve well between an off position, in which the valve assembly is in an off/closed configuration, and an on position, in which the valve assembly is in an on/open configuration. In the off configuration, the valve assembly blocks fluid communication between the fluid/suction source and the insertion tube of the endoscope. When the valve assembly is shifted into an on configuration (typically by being depressed toward the handle), fluid communication between the fluid/suction source and the working channel of the endoscope is established to supply fluid and/or to apply suction/negative pressure to the insertion tube of the endoscope.

Typically, a suction source coupled to an endoscope is continuously running during a procedure. However, it is generally desirable to limit application of suction during a procedure. For instance, in certain endoscopic procedures, it is desirable to maintain an anatomical site insufflated to improve visualization of the target site of the procedure, and/or to irrigate a target site, such as by supplying fluid to the target site. In such instances, the valve assembly is typically biased into an off configuration. Application of suction may be limited to reducing the supplied fluid in certain instances, and/or to remove other materials (e.g., biological materials) from the target site. To apply suction, the medical professional must actively depress the valve actuator, which is otherwise biased into an off position when in a neutral configuration (without application of an actuating force thereto). This can create fatigue during long procedures requiring suction regularly, even if intermittently. There remains a need for improvements to endoscope valves, such as actuators for suction valves.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, an actuatable member for a valve assembly of a medical instrument has a proximal end and a distal end, and further includes a user-engagement element along the proximal end thereof; and a shaft along the distal end thereof. In some aspects, the shaft is positionable within a valve well of the valve assembly and shiftable in the valve well along an actuation axis and between an on position in which the valve assembly is in an on configuration, and an off position in which the valve assembly is in an off configuration; and the actuatable member is held in each of the on position and the off position without an actuation force being applied thereto.

In some aspects, the actuatable member is held in each of the on position and the off position by an actuation component on one of the shaft or the user-engagement element.

In some aspects. the user-engagement element is movable with respect to the shaft. In some aspects, the user-engagement element and the shaft rotate with respect to each other to shift the shaft between the on position and the off position. In some aspects, the user-engagement element moves axially along the actuation axis and with respect to the shaft. In some aspects, the user-engagement element and the shaft together move axially along the actuation axis.

In some aspects, the shaft includes one of a cam surface or a cam follower configured to effect movement of the shaft between the on position and the off position upon engagement with the other of a cam surface or a cam follower associated with the valve assembly. In some aspects, the shaft includes a proximal cam surface and a distal cam surface each extending circumferentially around the shaft. In some aspects, the user-engagement element includes a radially-inwardly directed cam follower engaging the cam surfaces of the shaft to rotate the shaft between the on position and the off position. In some aspects, the cam follower holds the cam surfaces alternately in the on position or the off position. In some aspects, the shaft includes one of a movable cam follower or a vertically-extending cam surface having an on rest position for the cam follower in which the cam follower holds the shaft in the on position, and an off position for the cam follower in which the cam follower holds the shaft in the off position.

In some aspects, the shaft rotates between the on position and the off position.

In some aspects, the shaft axially shifts between the on position and the off position.

In some aspects, the actuatable member further includes a biasing element positioned to bias the user-engagement element proximally to a neutral position, the neutral position alternating between the on position and the off position upon sequential application and removal of a distal actuation force to the user-engagement element.

In accordance with various principles of the present disclosure, an actuatable member assembly for a valve assembly of a medical instrument includes an actuatable member with a user-engagement element along a proximal end thereof, and a shaft along a distal end thereof; and a collar extending circumferentially around the shaft and configured to be operatively engaged with the valve assembly to mount the actuatable member with respect to the valve assembly. In some aspects, the shaft is positionable within a valve well of the valve assembly and shiftable in the valve well along an actuation axis and between an on position in which the valve assembly is in an on configuration, and an off position in which the valve assembly is in an off configuration; and one of the user-engagement element, the shaft, or the collar includes a cam surface, and another of the user-engagement element, the shaft, or the collar includes a cam follower configured to engage the cam surface to effect movement of the shaft between the on position and the off position.

In some aspects, the actuatable member assembly further includes a biasing element positioned to bias the user-engagement element proximally to a neutral position, the neutral position alternating between the on position and the off position upon sequential application and removal of a distal actuation force to the user-engagement element to move the cam follower along the cam surface.

In accordance with various principles of the present disclosure, a method of actuating a valve assembly of a medical device includes applying an actuation force to an actuatable member of the valve assembly and releasing the actuation force, leaving the valve assembly in one of an on configuration or an off configuration; and applying an additional actuation force to the actuatable member and releasing the actuation force, leaving the valve assembly in the other of an on configuration or an off configuration.

In some aspects, the actuatable member is in a neutral position without application of an actuation force thereto, and a biasing element biases the actuatable member back to the neutral position upon release of an actuation force.

In some aspects, application of an actuation force to the actuatable member commences shifting of the valve assembly from one of an on configuration or an off configuration to the other of an on configuration or an off configuration, and release of the actuation force allows the biasing element to complete shifting of the valve assembly from one of an on configuration or an off configuration to the other of an on configuration or an off configuration so that the valve assembly remains in the other of an on configuration or an off configuration when the actuatable member returns to its neutral position.

In some aspects, releasing the actuation force allows the valve assembly to shift from one of an on configuration or an off configuration to the other of an on configuration or an off configuration.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an endoscope with one or more valves formed in accordance with various aspects of the present disclosure.

FIG. 2 illustrates a perspective view of an example of an embodiment of a valve assembly actuatable member formed in accordance with various principles of the present disclosure, and configured for mounting with respect to an endoscope such as illustrated in FIG. 1.

FIG. 3A illustrates a cross-sectional view of an example of an embodiment of a valve assembly with an actuatable member such as illustrated along line IIIA-IIIA of FIG. 2 positioned so that the valve assembly is in an off configuration.

FIG. 3B illustrates a view similar to that of FIG. 3A, but with the actuatable member pressed distally.

FIG. 3C illustrates a view similar to that of FIG. 3B, but with the actuatable member pressed further distally.

FIG. 3D illustrates a view similar to that of FIG. 3C, but with the actuatable member released from the position in FIG. 3C and returned toward a proximal position.

FIG. 3E illustrates a view similar to that of FIG. 3C, but with the actuatable member fully released to return to a neutral position such as illustrated in FIG. 3A, but in an on position with the valve assembly in an on configuration.

FIG. 4 illustrates a bottom perspective view of an example of an embodiment of a collar element such as in FIG. 2.

FIG. 5 illustrates an elevational view of an example of an embodiment of a valve assembly actuatable member formed in accordance with various principles of the present disclosure, mounted with respect to an example of an embodiment of a valve well and valve collar illustrated in cross section, and configured for mounting with respect to an endoscope such as illustrated in FIG. 1.

FIG. 6A illustrates an elevational view of an example of an embodiment of a valve assembly with an actuatable member such as illustrated in FIG. 5 with a valve port positioned so that the valve assembly is in an off configuration.

FIG. 6B illustrates a view similar to that of FIG. 6A, but with the actuatable member pressed distally.

FIG. 6C illustrates a view similar to that of FIG. 6B, but with the actuatable member pressed further distally.

FIG. 6D illustrates a view similar to that of FIG. 6C, but with the actuatable member released from the position in FIG. 6C and returned toward a proximal position.

FIG. 6E illustrates a view similar to that of FIG. 6D, but with the actuatable member released further from the position in FIG. 6C and returned an initial position as in FIG. 6A, but with the valve port now positioned so that the valve assembly is in an on configuration.

FIG. 7A illustrates an elevational view of an example of an embodiment of a valve assembly actuatable member formed in accordance with various principles of the present disclosure, mounted with respect to an example of an embodiment of a valve well and valve collar illustrated in cross section, and configured for mounting with respect to an endoscope such as illustrated in FIG. 1.

FIG. 7B illustrates an elevational view similar to that of FIG. 7A, but with a modified actuation mechanism component.

FIG. 8A illustrates an elevational view of an example of an embodiment of a valve assembly with an actuatable member such as illustrated in FIG. 7A with a valve port positioned so that the valve assembly is in an off configuration and the actuatable member is in a first stable position.

FIG. 8B illustrates a view similar to that of FIG. 8A, but with the actuatable member pressed distally.

FIG. 8C illustrates a view similar to that of FIG. 8B, but with the actuatable member released from the position of FIG. 8B and in a second stable position.

FIG. 8D illustrates a view similar to that of FIG. 8C, but with the actuatable member pressed distally to be released from the second stable position of FIG. 8C to return to the first stable position of FIG. 8A.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, etc.) such location or site.

Various medical devices include valve assemblies to regulate or control fluid delivery (irrigation) or fluid suction (aspiration) with respect to an anatomical site. Although the present disclosure describes suction valves, it will be appreciated that the principles of the present disclosure need not be so limited.

A suction valve assembly of a medical device is arranged to apply suction from a suction source to an anatomical site, such as via a flexible tubular element which is configured and positionable with respect to the anatomical site. The suction source may be a pump or other mechanism creating a vacuum to be applied to the anatomical site via the flexible tubular element. In the off configuration of the valve assembly, fluid communication between the suction source and the flexible tubular element is cut off or blocked so that suction is not applied to the anatomical site, and the valve may be considered to be in a closed configuration. In the on configuration of the valve assembly, the suction source is fluidly coupled with the flexible tubular element, such as to aspirate an anatomical site, and the valve may be considered to be in an open configuration.

Valve assemblies of medical devices typically include an actuatable member movable, along an actuation axis, within a valve well to shift the valve assembly between an off position placing the valve assembly in an off configuration, and an on position placing the valve assembly in on configuration. The actuatable member may include a user-engagement element and a valve shaft. Various valve assemblies have different arrangements of ports and flow paths placing a fluid source, such as a suction source, in and out of fluid communication with an anatomical site. For instance, in some valve assemblies, the fluid/suction source is fluidly coupled with a source port in the valve well which extends generally transverse to the actuation axis of the valve shaft. In such valve assemblies, the application port (via which a fluid/suction application device applies fluid/suction to an anatomical site) is generally axially aligned with the actuation axis of the valve shaft. In other valve assemblies, the fluid/suction source is fluidly coupled with a source port in the valve well which is generally axially aligned with the actuation axis of the valve shaft. In such valve assemblies, the application port in the valve well extends transverse to the actuation axis of the valve shaft. Principles of the present disclosure may be applied to either configuration of a valve assembly. In either configuration, a valve well channel extends through the valve well to fluidly communicate the axial port and the transverse port of valve well. The actuatable member of the valve assembly is movably mounted within such valve well channel to axially and/or rotationally shift between on and off positions to shift the valve assembly between respective on and off configurations. The actuatable member has an axial flow passage therethrough (extending generally along the actuation axis of the actuatable member) and/or a transverse flow passage therethrough. The flow passages through the actuatable member fluidly couple the source port and the application port of the valve well when the valve shaft is in the on position. The actuatable member blocks fluid communication between the source port and the application port of the valve well when the valve shaft is in the off position.

Principles of the present disclosure are described. for the sake of convenience, with respect to a valve assembly having a supply port fluidly coupled to a suction source, and a suction application port fluidly coupled with a suction application device. However, it will be appreciated that principles of the present disclosure are applicable to valve assemblies other than those configured to apply suction. The suction source may be a pump or any other mechanism capable of creating a vacuum, such as known to those of ordinary skill in the art. The suction application device may be any tubular element capable of applying suction from the suction source to an anatomical site, such as an insertion tube of an endoscope (e.g., with a suction lumen and/or working channel therethrough).

In accordance with various principles of the present disclosure, instead of the actuatable member being biased into an off position, as in prior art valve assemblies (particularly suction valve assemblies), the actuatable member is stable in the on position as well as in the off position, and is actively actuated to move between such stable on or off positions. As used herein, the term “stable” as used with reference to a position connotes that such position is independently maintained and does not shift without application of force thereto. In other words, the actuatable member is held in each of the on position and the off position without an actuation force being applied thereto. The actuatable member may be in one of the on position or the off position until actively actuated to move to the other of the on position or the off position, and then remains in such other position until actively actuated again to shift back to the one of the on position or the off position. For instance, the actuatable member may be in an on position until actively actuated to shift to an off position, and then remains in the off position until actively actuated to move to the on position. Once returned to the on position, the actuatable member remains in the on position until actively actuated by a user to move to the off position again. References to active actuation, and the like, are to be understood herein as actuation (e.g., movement) by a user (e.g., a medical professional) upon application of an actuation force (e.g., an external force, such as an intentional actuation force, typically applied by depressing the actuatable member in a direction towards the housing/handle with respect to which the valve assembly is mounted), in contrast with current valve assemblies which automatically return to the same position.

In accordance with various principles of the present disclosure, a valve assembly includes an actuatable member movable with respect to a valve well. More particularly, in some embodiments, the actuatable member has a valve shaft with ports and flow channels shiftable into and out fluid communication with ports in the valve well to shift the valve assembly between on and off configurations. For instance, an example of an embodiment of a valve shaft formed in accordance with various principles of the present disclosure has a transversely-extending port and an axially-extending port fluidly coupled via a flow channel (e.g., axially extending therebetween). The valve shaft transversely-extending port is movable into and out of fluid communication with a transversely-extending port defined in the valve well. In an on position of the valve shaft, the valve shaft transversely-extending port is in fluid communication with the valve well transversely-extending port, thereby placing the valve well transversely-extending port in fluid communication with the valve well axially-extending port via the valve shaft flow channel and axially-extending port. Such configuration of the valve assembly is considered an on configuration. In an off position of the valve shaft, the valve shaft transversely-extending port is not in fluid communication with the valve well transversely-extending port, and the valve well transversely-extending port is no longer in fluid communication with the valve well axially-extending port. Such configuration of the valve assembly is considered an off configuration.

In some embodiments, axial movement of the actuatable member causes rotational movement of the valve shaft to shift the valve shaft between off and on positions. In some embodiments, axial movement of the actuatable member causes axial movement of the valve shaft to shift the valve shaft between off and on positions. In some embodiments, axial movement of the actuatable member causes both rotational and axial movement of the valve shaft to shift the valve shaft between off and on positions.

The actuatable member is held in one of an on or off position until actuated into the other of the on or off position.

In accordance with various principles of the present disclosure, a portion of the actuatable member includes an actuation component configured to engage an actuation component on another portion of the valve assembly to cause shifting of the actuatable member between an on position and an off position. For instance, in some embodiments, the valve shaft of the actuatable member includes an actuation component operatively engaging an actuation component on the user-engagement element of the actuatable member or a collar component of the valve assembly. Application of an actuation force to the actuatable member causes the actuation components to operatively engage each other to cause shifting of the position of the actuatable member between on and off configurations. Moreover, the actuatable member is configured to remain in an on configuration as well as to remain in an off configuration until an actuation force is applied thereto. In accordance with various principles of the present disclosure, an actuation component on a component of the actuatable member maintains the actuatable member in an on position and also maintains the actuatable member in an off position. As such, the valve assembly is maintained in an on configuration or an off configuration without the need to apply a continuous force to the actuatable member to maintain the valve assembly in a selected configuration.

Various embodiments of valve devices (including, without limitation, components and assemblies), systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments of valve assemblies and associated components are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

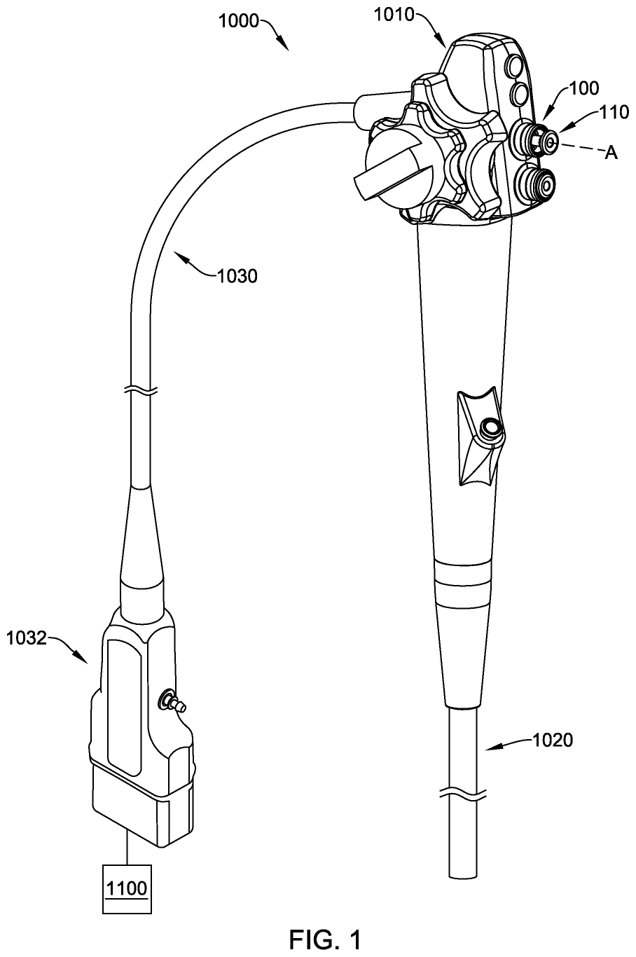

Turning now to the drawings, an example of an embodiment of a valve assembly 100 formed in accordance with various principles of the present disclosure is illustrated in FIG. 1 as provided in an example of an embodiment of an endoscope 1000. It will be appreciated that the endoscope 1000 is an example of an embodiment in which principles of the present disclosure may be applied, and that various principles of the present disclosure are applicable to other medical instruments to control fluid flow with respect thereto, the details of which are not critical to the present disclosure. Moreover, although reference is made to a suction valve, it will be appreciated that the disclosed principles and embodiments are applicable to other valves, such as fluid supply/irrigation valves.

The illustrated example of an embodiment of a valve assembly 100 is mounted with respect to a control handle 1010 of the endoscope 1000 to regulate the flow of materials (e.g., fluid) between the insertion tube 1020 of the endoscope 1000 and a suction source 1100. The endoscope 1000 has a connector cord 1030 extending to a scope connector 1032 with which the endoscope 1000 (and valve assembly 100) may be fluidly coupled with the suction source 1100. The connector cord 1030 may be alternatively referenced herein as an umbilical cord, umbilicus, universal cord, etc., without intent to limit. The scope connector 1032 may also couple the endoscope 1000, via the connector cord 1030, with a variety of components, devices, etc., such as a fluid source (to supply air, carbon dioxide, water, saline, or other gases or liquids), electrical connections, light sources, visualization elements (e.g., optic fibers, cameras, etc.), or other components, devices, etc., usable with the endoscope 1000. The insertion tube 1020 has a fluid lumen extending therethrough to a distal end which is positionable (insertable, navigable, etc.) with respect to an anatomical site (e.g., within a patient). Similarly, the connector cord 1030 has a fluid lumen extending therethrough to fluidly couple the suction source 1100 (e.g., via the scope connector 1032) with the control handle 1010. The fluid lumens through the insertion tube 1020 and the connector cord 1030, and the distal end of the insertion tube 1020, may be well-known features formed in a manner known to those of ordinary skill in the art and are not illustrated to simplify the drawings by eliminating details in the illustration of the endoscope 1000 in FIG. 1 which are not necessary for understanding the present disclosure.

The valve assembly 100 has an actuatable member 110 configured to be shifted between an on position and an off position, along an actuation axis A, upon application of an actuation force thereto. Typically, the actuation force is applied in a distal direction, such as towards the control handle 1010 housing the valve assembly 100, and typically along the actuation axis A, by a user of the valve assembly 100, such as a medical professional. When the valve assembly 100 is in an on configuration, a source port and an application port of the valve assembly 100 are in fluid communication. For instance, with reference to the example of an embodiment illustrated in FIG. 1, a suction source 1100 may be fluidly coupled with an insertion tube 1020 of the endoscope 1000 to apply suction when the valve assembly 100 is in the on configuration. When the valve assembly 100 is in an off configuration. the source port and the application port of the valve assembly 100 are not in fluid communication (and typically are sealed with respect to each other). For instance, with reference to the example of an embodiment illustrated in FIG. 1, the endoscope 1000 does not apply suction when the valve assembly 100 is in an off configuration.

Further in accordance with various principles of the present disclosure, upon removal the actuation force applied to change the configuration of the valve assembly 100 from one of the on or off configurations to the other of the on or off configurations, the valve assembly 100 remains in the other configuration. In other words, if an actuation force is applied to the actuatable member when the valve assembly 100 is in an on configuration to change the configuration of the valve assembly 100 to an off configuration, then the valve assembly 100 remains in the off configuration once the actuation force is no longer applied to the actuatable member. Conversely, if an actuation force is applied to the actuatable member when the valve assembly 100 is in an off configuration to change the configuration of the valve assembly 100 to an on configuration, then the valve assembly 100 remains in the on configuration once the actuation force is no longer applied to the actuatable member. The actuatable member 110 thus has a first stable position in which the actuatable member 110 is in one of an on or off position, placing the valve assembly 100 in a corresponding on or off position, and a second stable position in which the actuatable member 110 is in the other of an on or off position, placing the valve assembly 100 in the corresponding other of an on or off position.

The actuatable member 110 of a valve assembly 100 may have ports corresponding with ports within a valve well of the valve assembly 100 and movable in and out of fluid communication with the valve well ports to shift the valve assembly 100 between on and off configurations. In accordance with various principles of the present disclosure, at least one component of an actuatable member 110 includes an actuation mechanism configured to shift a port defined in the actuatable member 110 in and out of fluid communication with a port in another component of the valve assembly 100, such as a port in a valve well of the valve assembly 100. The component of the actuatable member 110 with the port may be axially movable along the actuation axis A, or rotatable about the actuation axis A, or both axially movable as well as rotatable about the actuation axis A to shift the actuatable member 110 between on and off positions with the port of the actuatable member 110 in or out of fluid communication with a port of the valve well of the valve assembly 100. The actuation mechanism includes an actuation component operatively engageable with another actuation component on the actuatable member 110 or another component of the valve assembly 100. The actuation components may be cam followers and/or cam surfaces such as described below.

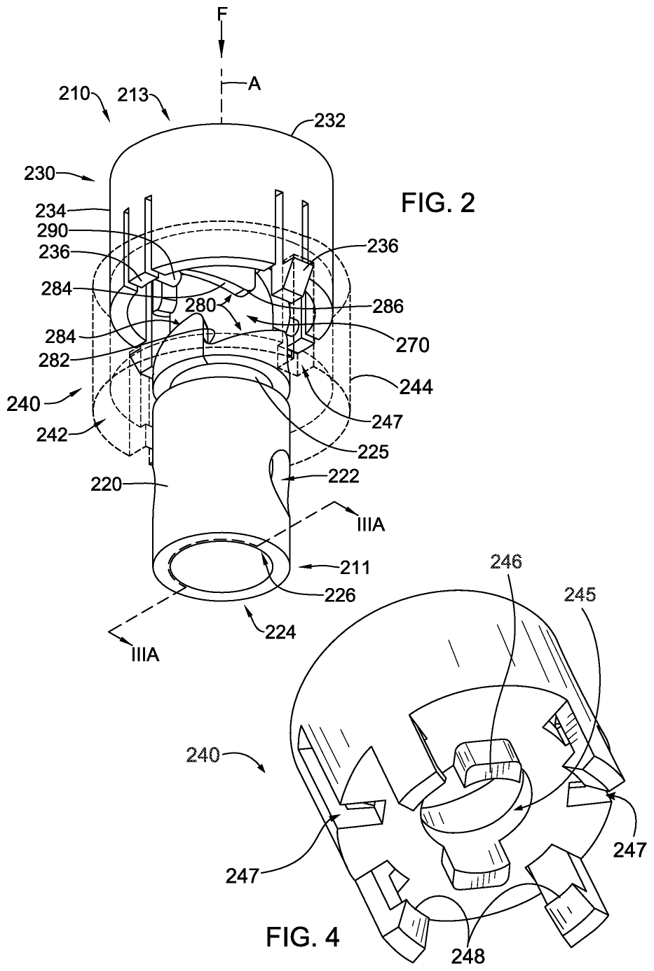

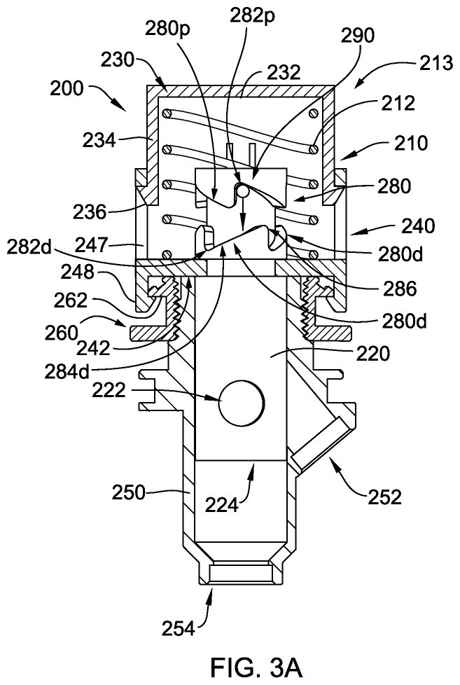

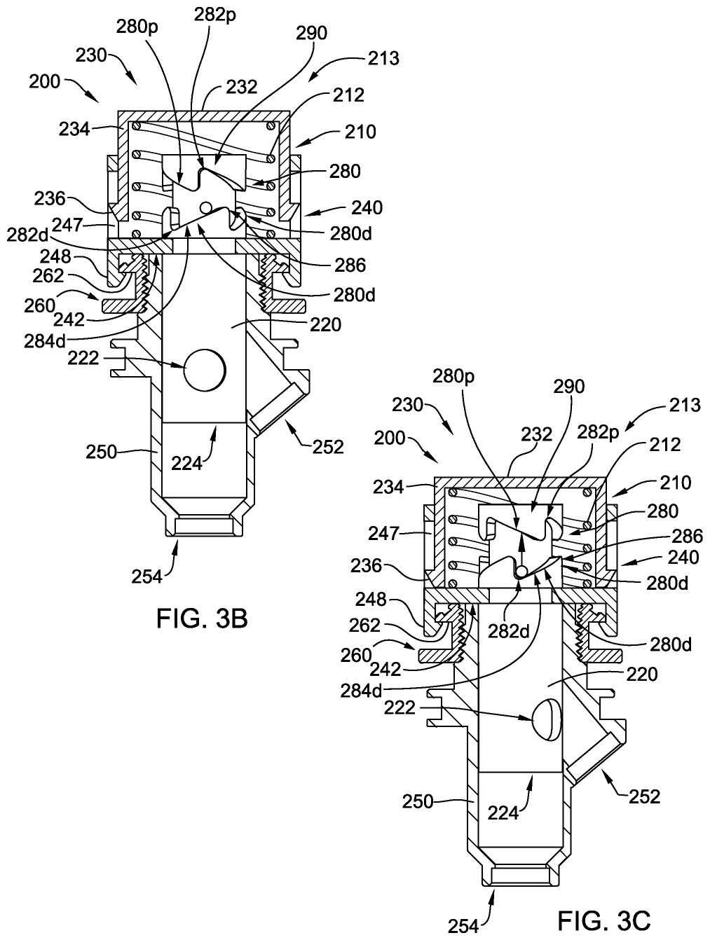

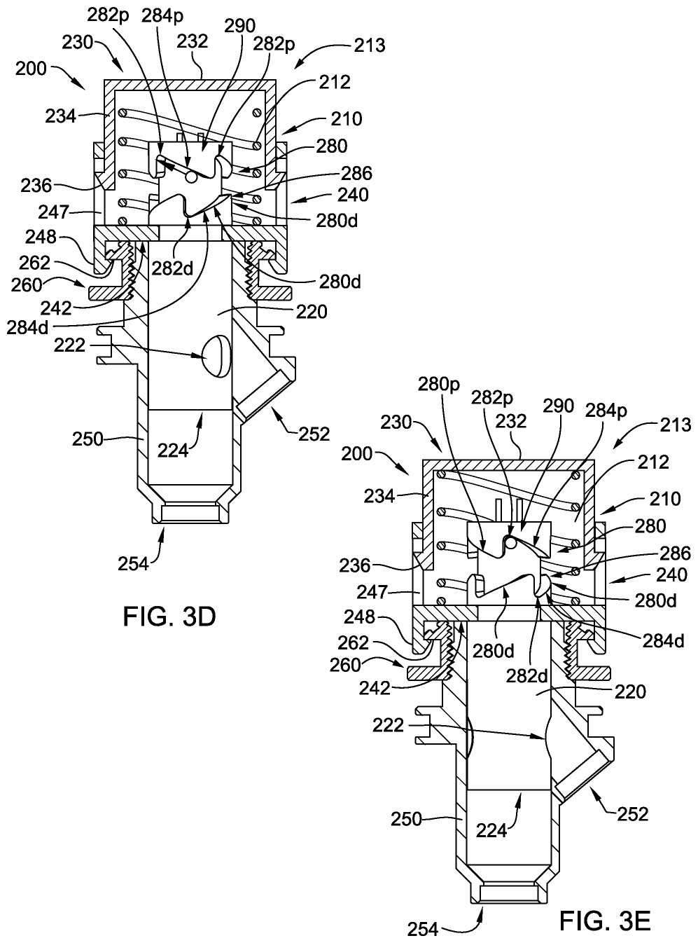

An example of an embodiment of an actuatable member 210 of a valve assembly 200 formed in accordance with various principles of the present disclosure is illustrated in FIG. 2, in isolation from an endoscope (such as the endoscope 1000 illustrated in FIG. 1). The illustrated example of an embodiment of an actuatable member 210 includes a valve shaft 220 and a user-engagement element 230, which are operatively coupled together. The valve shaft 220 generally extends along the distal end 211 of the actuatable member 210, and the user-engagement element 230 generally extends along the proximal end 213 of the actuatable member 210. The valve shaft 220 and the user-engagement element 230 are movable with respect to each other, generally axially as well as rotationally with respect to the actuation axis A. A biasing element 212 may be positioned with respect to the valve shaft 220 and the user-engagement element 230 to maintain the user-engagement element 230 spaced apart from the valve shaft 220 in a neutral configuration, as illustrated in FIG. 3A.In the example of an embodiment illustrated in FIG. 2 and FIGS. 3A-3E, a collar 240 is mounted around the valve shaft 220 and the user-engagement element 230, and may maintain the relative positions of the valve shaft 220 and the user-engagement element 230 with respect to each other as well as with respect to a valve well 250 of the valve assembly 200. For instance, as illustrated in FIGS. 3A-3E, the collar 240 has a shaft retainer wall 242 extending radially-inwardly from a generally cylindrical skirt 244 of the collar 240. The biasing element 212 may be positioned between the shaft retainer wall 242 and the user-engagement element 230 to bias the user-engagement element 230 away from the valve shaft 220. Additionally, the shaft retainer wall 242 defines a shaft-retaining opening 245 (as shown in FIG. 4) through which the valve shaft 220 is extended. The valve shaft 220 may include a circumferential groove 225 (illustrated, for example, in FIG. 2) engaged with respect to the shaft-retaining opening 245 of the collar 240, such as to limit axial movement of the valve shaft 220 with respect to the collar 240. The collar skirt 244 defines an axially-extending slot 247. The user-engagement element 230 has a skirt 234 (distally extending from the user-engagement surface 232), axially movable with respect to and partially within the collar skirt 244, with a radially-outwardly extending projection 236 configured to extend into a corresponding axially-extending slot 247 defined in the collar skirt 244 to limit movement of the user-engagement element 230 with respect to the collar 240 to axial movement along the actuation axis A.

In accordance with various principles of the present disclosure, axial movement of the actuatable member 210 along the actuation axis A causes rotational movement of the valve shaft 220 with respect to the user-engagement element 230 and with respect to the valve well 250 to shift the transversely-extending valve shaft port 222 (extending in a direction transverse to the actuation axis A) defined in the valve shaft 220 between an on position and an off position. In the on position of the valve shaft 220, the valve shaft port 222 is fluidly coupled and generally aligned with a transversely-extending valve well port 252 defined in the valve well 250. In the off position of the valve shaft 220, the valve shaft port 222 is not in fluid communication with (and typically sealed from) the transversely-extending valve well port 252 defined in the valve well 250. The transversely-extending port 222 of the valve shaft 220 is in fluid communication with an axially-extending port 224 via a valve shaft channel 226 extending generally axially through the valve shaft 220. The axially-extending port 224 of the valve shaft 220 is in fluid communication with an axially-extending port 254 of the valve well 250. Thus, when the valve shaft transversely-extending port 222 is in fluid communication with the valve well transversely-extending port 252, the valve well transversely-extending port 252 is in fluid communication with the valve well axially-extending port 254, thereby allowing suction to be applied through the valve assembly 200.

In the example of an embodiment illustrated in FIG. 2 and FIGS. 3A-3E, application of an actuation force F to the user-engagement element 230 of the actuatable member 210 (e.g., to a user-engagement surface 232 thereof) in a distal direction (towards the distal end 211 of the actuatable member 210) causes axial movement, along the actuation axis A, of the user-engagement element 230 which is transformed into rotational movement of the valve shaft 220 about the actuation axis A. Rotation of the valve shaft 220 shifts the valve shaft 220 with respect to the valve well 250 from one of an on/off position to the other of an on/off position. Unlike prior valve assemblies which return to an off position upon removal of an actuation force applied to an actuatable member thereof, in accordance with various principles of the present disclosure, the valve shaft 220 remains in the on position or in the off position upon removal of the actuation force F which has placed the valve shaft 220 in such position.

In order to effect such relative rotational movement between the valve shaft 220 and the valve well 250, the user-engagement element 230 is fixed against rotating with respect to the valve well 250 of the valve assembly 200, while the valve shaft 220 rotates with respect to the valve well 250. In the example of an embodiment of a valve assembly 200 illustrated in FIG. 2, FIGS. 3A-3E, and FIG. 4, the collar 240 which is positioned around the valve shaft 220 and the user-engagement element 230 is mounted to be rotationally fixed with respect to the valve well 250 within the valve assembly 200 (illustrated in FIGS. 3A-3E). Since the user-engagement element 230 is rotationally fixed with respect to the collar 240, as described above, the user-engagement element 230 thereby fixed rotationally with respect to the valve well 250. Since the valve shaft 220 is rotatable with respect to the user-engagement element 230 and the collar 240, the valve shaft 220 is rotatable within the valve well 250. The collar 240 may be mounted with respect to the valve well 250 to be rotationally fixed thereto in any of a variety of manners, such as known to those of ordinary skill in the art. For instance, the collar 240 may include axially-extending projections 246 (as shown in FIG. 4) configured to engage corresponding seats in the valve well 250 (not shown, but which may be in a configuration known to those of ordinary skill in the art) to lock the collar 240 rotationally with respect to the valve well 250. The collar 240 may further includes one or more projection 248 extending radially-inwardly from the collar skirt 244 configured to engage with the valve well 250 to mount the collar 240 with respect thereto. In the example of an embodiment illustrated in FIGS. 3A-3E, the collar 240 is mounted to the valve well 250 via an optional valve well nut 260 (which, in turn, is coupled, e.g., threadedly coupled, with respect to the valve well 250). The radially-inwardly extending projection 248 on the skirt 244 of the collar 240 engage a radially-outwardly extending circumferential flange 262 on the valve well nut 260 to mount the collar 240 with respect to the valve well nut 260 and thus to the valve well 250 on which the valve well nut 260 is mounted. As such, rotation of the collar 240 with respect to the valve well 250 is inhibited, and rotation of the user-engagement element 230 with respect to the collar 240 is inhibited, while the valve shaft 220 is rotatable with respect to the valve well 250.

In the example of an embodiment illustrated in FIG. 2, FIGS. 3A-3E, and FIG. 4, to actuate the valve shaft 220 to rotate with respect to the valve well 250, an actuation mechanism 270 is positioned with respect to the valve shaft 220 and the user-engagement element 230 and configured to convert the axial movement of the user-engagement element 230 along an actuation axis A into rotational movement of the valve shaft 220. As the valve shaft 220 rotates, the transversely-extending valve shaft port 222 is rotated sequentially into and out of fluid communication with the transversely-extending valve well port 252 in the valve well 250, shifting the valve assembly 200 sequentially between on and off configurations, as illustrated in FIGS. 3A-3E, and described in further detail below. In accordance with various principles of the present disclosure, the valve shaft 220 remains in a selected position (either the on position or the off position) when an actuation force is not applied to the actuatable member 210.

An example of an embodiment of an actuation mechanism 270 comprising a cam surface 280 and a corresponding cam follower 290 configured to convert axial movement of the user-engagement element 230 into rotational movement of the valve shaft 220 is illustrated in FIG. 2 and in FIGS. 3A-3E. In the illustrated example of an embodiment, the cam surface 280 (which includes a proximal cam surface 280p and a distal cam surface 280d) extends circumferentially around the outer surface of the valve shaft 220, and the cam follower 290 extends radially-inwardly from the skirt 234 of the user-engagement element 230. However, a reverse arrangement, with a cam follower extending radially outwardly from the valve shaft 220 and riding along a cam surface along the interior surface of the skirt 234 of the user-engagement element 230, is within the scope of the present disclosure as well.

Operation of an actuation mechanism 270 formed in accordance with various principles of the present disclosure may be appreciated with reference to the sequential positions of the cam follower 290 with respect to the cam surface 280 illustrated in FIGS. 3A-3E (with arrows in FIG. 3A-3E indicating the next movement of the cam follower 290). The valve assembly 200 is illustrated in an off configuration in FIG. 3A, with the valve shaft 220 in an off position with the transversely-extending valve shaft port 222 not aligned with and not fluidly communicating with the transversely-extending valve well port 252. Moreover, the actuatable member 210 is in a neutral configuration in FIG. 3A, with no actuation force being applied thereto. In a neutral configuration of the actuatable member 210, the cam follower 290 of the actuation mechanism 270 rests in a proximal valley 282p of the proximal cam surface 280p, as illustrated in FIG. 3A. As may be appreciated, the proximal valleys 282p of the proximal cam surfaces 280p define a rest position for the cam follower 290. As such, when the cam follower 290 is positioned in a proximal valley 282p, the cam follower 290 remains in place unless or until an actuation force F is applied to the user-engagement element 230 to move the cam follower 290 out of the proximal valley 282p. The proximal valley 282p may also be considered to define a proximal limit stop for proximal movement of the user-engagement element 230.

When an actuation force F is applied to the user-engagement element 230 to activate the actuation mechanism 270, the cam follower 290 moves distally with the user-engagement element 230 and engages a distal sloped surface 284d of the distal cam surface 280d, as illustrated in FIG. 3B. The sloped surfaces 284 of the cam surface 280 extend transverse to a plane perpendicular to the actuation axis A and between a valley 282 and a peak 286 of the cam surface 280. Since the cam follower 290 is restrained from rotational movement (because the cam follower 290 extends from the user-engagement element 230 which is rotationally fixed with respect to the collar 240, which, in turn, is rotationally fixed with respect to the valve well 250, as described above), further axial movement of the cam follower 290 along a sloped surface 284 of the cam surface 280 causes rotation of the valve shaft 220. More particularly, further distal axial movement of the user-engagement element 230 with respect to the valve shaft 220 causes rotation of the valve shaft 220 in a counterclockwise direction when viewed from the proximal end 213 of the actuatable member 210, or to the right in FIG. 3B. As the valve shaft 220 rotates, the cam follower 290 rides along the distal sloped surface 284d of the distal cam surface 280d to rest in a distal valley 282d of the distal cam surface 280d, as illustrated in FIG. 3C. The distal valley 282d may be considered to define a distal limit stop for distal movement of the user-engagement element 230. As may be appreciated upon comparison of FIG. 3B and FIG. 3C, rotation of the valve shaft 220 also rotates the transversely-extending valve shaft port 222 closer to the transversely-extending valve well port 252. As may be appreciated with reference to FIG. 2, each of the proximal cam surface 280p and the distal cam surface 280d includes four sets of valleys 282, sloped surfaces 284, and peaks 286 extending around the valve shaft 220. As such, as the cam follower 290 moves from a position generally mid-way along the distal sloped surface 284d (as illustrated in FIG. 3B) to a distal valley 282d (as illustrated in FIG. 3C), the valve shaft 220 rotates ⅛ of a turn (45°) about the actuation axis A.

Removal of the actuation force F from the user-engagement element 230 allows the biasing element 214 to move the user-engagement element 230 proximally, toward the proximal end 213 of the actuatable member 210, as illustrated in FIG. 3D. As described above with reference to FIG. 3B, but in the reverse direction, the cam follower 290 moves proximally with the user-engagement element 230 and engages a proximal sloped surface 284p of the proximal cam surface 280p, as illustrated in FIG. 3D. As described above with respect to FIG. 3B and FIG. 3C, constraint of the cam follower 290 to axial movement causes further axial movement of the cam follower 290 along a sloped surface 284 of the cam surface 280 to cause rotation of the valve shaft 220. More particularly, as may be appreciated with reference to FIG. 3D and FIG. 3E, further proximal axial movement of the user-engagement element 230 with respect to the valve shaft 220 causes the cam follower 290 to ride along the proximal sloped surface 284p of the proximal cam surface 280p and to rotate the valve shaft 220 counterclockwise (when viewed from the proximal end 213 of the actuatable member 210, or to the right in FIG. 3D) from the position illustrated in FIG. 3D to the position illustrated in FIG. 3E. The cam follower 290 thus moves from a position generally mid-way along the proximal sloped surface 284p (as illustrated in FIG. 3D) to a proximal valley 282p (as illustrated in FIG. 3E), rotating the valve shaft 220 another ⅛ of a turn (45°) about the actuation axis A. As may be appreciated upon comparison of FIG. 3D and FIG. 3E, rotation of the valve shaft 220 rotates the transversely-extending valve shaft port 222 closer to the transversely-extending valve well port 252. Moreover, as may be appreciated upon comparison of FIG. 3E and FIG. 3A, the sequence of movements of the actuatable member 210, from application of an actuation force F to the user-engagement element 230 to removal of such force (allowing the biasing element 212 to return to the actuatable member 210 to a neutral configuration), causes the transversely-extending valve shaft port 222 to move into fluid communication with the transversely-extending valve well port 252.

As may be appreciated, once the cam follower 290 is positioned in the proximal valley 282p (such as illustrated in FIG. 3A or in FIG. 3E), the cam follower 290 does not move, and thus the valve shaft 220 does not rotate until the actuatable member 210 is actuated. Thus, the valve shaft 220 remains in its position (either on or off) and the valve assembly 200 remains in its configuration (either on or off) until the actuatable member 210 is actuated. A user of the valve assembly 200 therefore need only apply an actuation force F to the actuatable member 210 for a limited time to shift the valve shaft 220 thereof to a different position to shift the valve assembly 200 to a different configuration. Once the actuation force F has been applied to shift the valve shaft 220 and the valve assembly 200, the actuation force F need not be continuously applied to maintain the valve shaft 220 and the valve assembly 200 in the position/configuration to which they have been shifted.

Instead of an actuation mechanism being positioned with respect to a valve shaft and a user-engagement element of an actuatable member to effect shifting of the valve shaft between an on position and an off position (to shift a valve assembly between an on configuration and an off configuration), an actuation mechanism may be positioned with respect to the actuatable member and the collar of a valve assembly to effect shifting of the valve shaft between an on position and an off position. As such, the user-engagement element may or may not move with respect to the valve shaft as the valve shaft moves to change the configuration of the valve assembly. In accordance with various principles of the present disclosure, the actuatable member of such embodiments has a neutral configuration such that the valve assembly remains in the configuration into which the actuation mechanism has shifted the valve assembly even if an actuation force is no longer applied to the actuatable member.

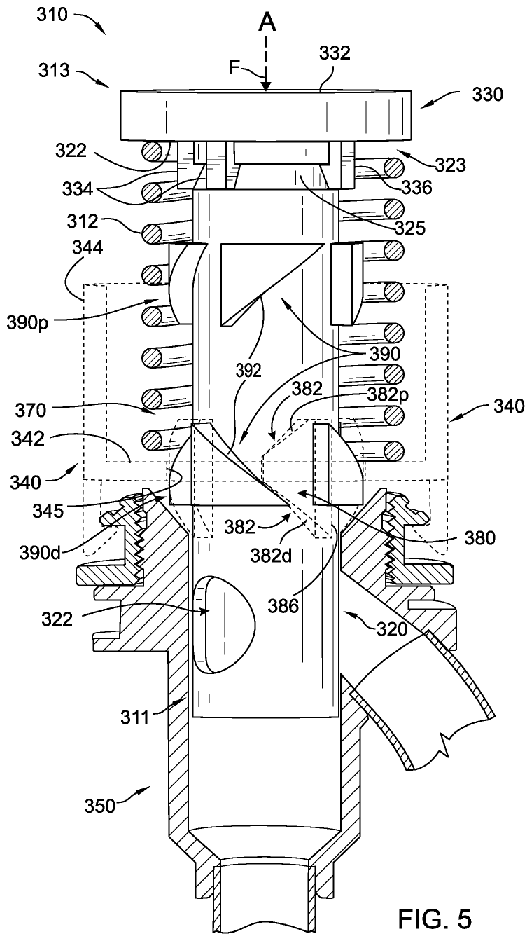

An example of an embodiment of an actuatable member 310 with an actuation mechanism 370 positioned with respect thereto and another component of a valve assembly is illustrated in FIG. 5. Like the example of an embodiment of an actuatable member 210 illustrated in FIG. 2, FIGS. 3A-3E, and FIG. 4, the actuatable member 310 illustrated in FIG. 5 includes a valve shaft 320 and a user-engagement element 330 which are operatively coupled together. The valve shaft 320 generally extends along the distal end 311 of the actuatable member 310, and the user-engagement element 330 generally extends along the proximal end 313 of the actuatable member 310. The user-engagement element 330 may be integrally formed with the valve shaft 320 (e.g., as a proximal end 323 thereof), or formed separately from the valve shaft 320 and optionally movable with respect thereto.

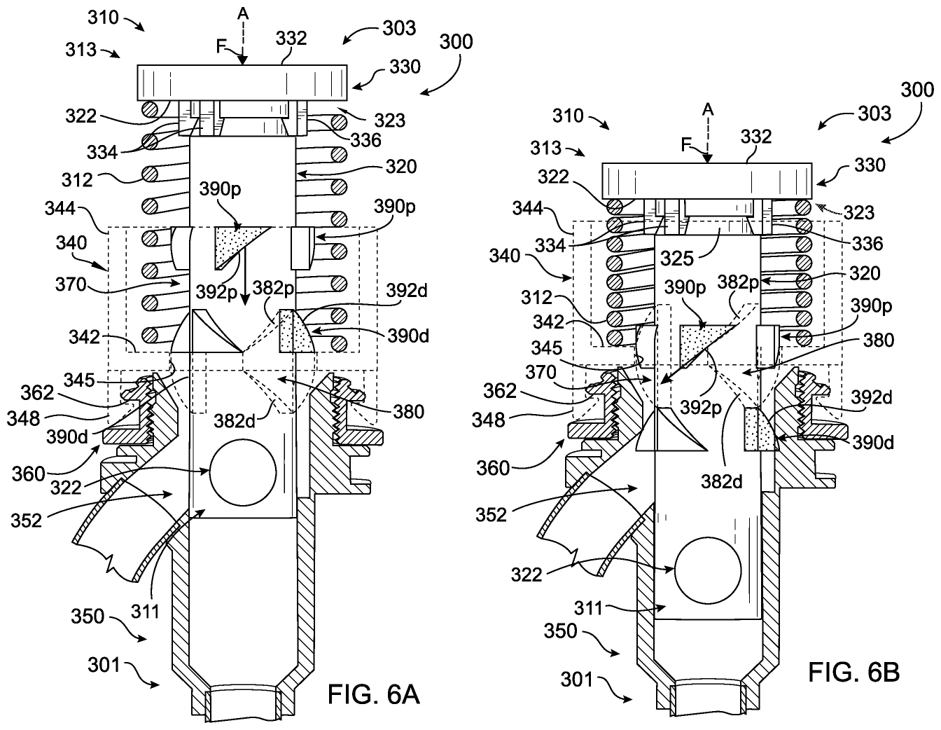

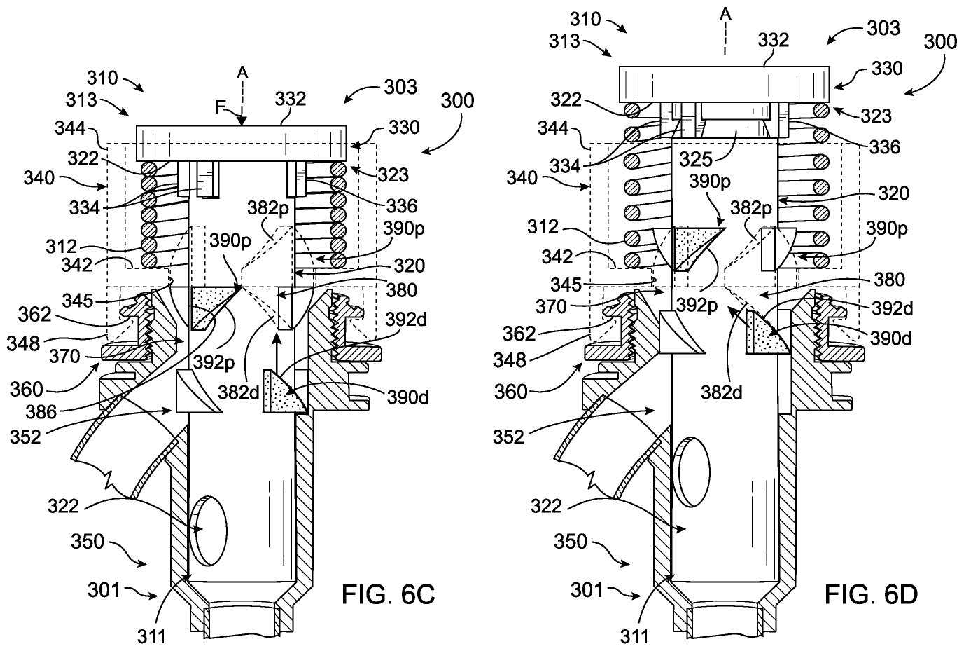

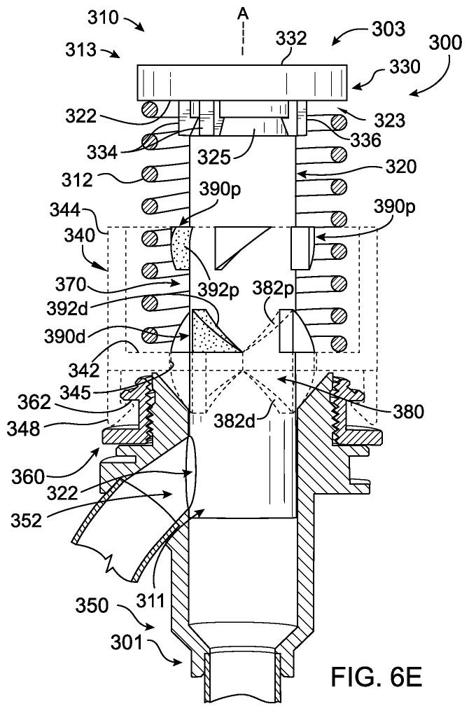

In accordance with various principles of the present disclosure, as illustrated in FIG. 5 and in FIGS. 6A-6E, a collar 340 is positioned around the actuatable member 310, and an actuation mechanism 370 is positioned with respect to the actuatable member 310 and the collar 340. The collar 340 is mounted with respect to a valve well 350 of a valve assembly 300 to be rotationally fixed with respect to the valve well 350, such as in a manner known to those of ordinary skill in the art. In the example of an embodiment illustrated in FIG. 5 and FIGS. 6A-6E, the collar 340 is mounted with respect to the valve well 350 in a manner such as illustrated in FIG. 2, FIGS. 3A-3E, and FIG. 4. For the sake of convenience, and without intent to limit, components illustrated in FIG. 5 and FIGS. 6A-6E which are similar to components illustrated in FIG. 2, FIGS. 3A-3E, and FIG. 4 are indicated with similar reference numbers increased by 100 and reference is made to the descriptions thereof in connection with FIGS. 3A-3E for the sake of brevity and without intent to limit.

The actuation mechanism 370 is configured to convert axial movement of the actuatable member 310 into rotational movement of the valve shaft 320. More particularly, distal and proximal axial movements of the actuatable member 310 along the actuation axis A engages a portion of the actuation mechanism 370 associated with the valve shaft 320 with a portion of the actuation mechanism 370 associated with the collar 340 to cause rotation of the valve shaft 320 with respect to the valve well 350, such as sequentially illustrated in FIGS. 6A-6E As the valve shaft 320 rotates, a transversely-extending valve shaft port 322 defined in the valve shaft 320 is rotated sequentially into and out of fluid communication with a transversely-extending valve well port 352 in the valve well 350 of the valve assembly 300, shifting the valve assembly 300 sequentially between on and off configurations, as illustrated in FIGS. 6A-6E, and as described in further detail below.

Optionally, the user-engagement element 330 is formed separately from and rotatable with respect to the valve shaft 320 so that axial movement of the user-engagement element 330 causes axial movement of the valve shaft 320 to actuate the actuation mechanism 370 to rotate the valve shaft 320 without rotating the user-engagement element 330 as well (such as for user comfort). For instance, the example of an embodiment of a user-engagement element 330 illustrated in FIG. 5 has a proximally-facing user-engagement surface 332 positioned along the proximal end 313 of the actuatable member 310, separately formed from and positioned over and covering a proximal end 323 of the valve shaft 320. Axial extensions 334 extend distally toward and around the valve shaft 320, with radially-inwardly directed projections 336 engaging with a circumferentially-extending groove 325 around the proximal end 323 of the valve shaft 320 to couple the user-engagement element 330 with the valve shaft 320, while optionally allowing relative rotational movement therebetween.

Application of an actuation force F to the user-engagement surface 332 of the user-engagement element 330 causes distal axial movement of the actuatable member 310 (toward the distal end 351 of the valve well 350) and actuation of the actuation mechanism 370 to rotate the valve shaft 320. Optionally, a biasing element 312 biases the user-engagement element 330 proximally upon removal of the actuation force F. The biasing element 312 may be positioned between the user-engagement element 330 (e.g., the underside thereof) and a shaft retainer wall 342 extending radially-inwardly from a generally cylindrical skirt 344 of the collar 340, and defining a shaft-retaining opening 345 through which the valve shaft 320 extends. Proximal movement of the actuatable member 310 (e.g., to its initial position prior to application of an actuation force F thereto) causes further actuation of the actuation mechanism 370 to complete the rotation of the valve shaft 320 to shift from one of an on or off position to the other of an on or off position. In accordance with various principles of the present disclosure, the valve shaft 320 remains in the other of the on or off position (the position to which the valve shaft 320 has been shifted by the actuation force F) when an actuation force is not applied to the actuatable member 310.

The example of an embodiment of an actuation mechanism 370 illustrated in FIG. 5 and FIGS. 6A-6D includes a cam surface 380 extending circumferentially along the interior surface of the generally cylindrical skirt 344 of the collar 340, and a cam follower 390 extending radially-outwardly from the outer surface of the valve shaft 320. However, a reverse arrangement, with a cam follower extending radially inwardly from the interior surface of the collar skirt 344 and riding along a cam surface extending circumferentially around the outer surface valve shaft 320, is within the scope of the present disclosure as well. More particularly, the example of an embodiment of a cam follower 390 illustrated in FIG. 5 and FIGS. 6A-6E includes a proximal cam follower 390p and a distal cam follower 390d axially spaced apart from each other, with the cam surface 380 extending therebetween. Even more particularly, in the example of an embodiment illustrated in FIG. 5 and FIGS. 6A-6E, the proximal cam follower 390p includes a plurality of proximal cam followers 390p circumferentially spaced apart from one another around the valve shaft 320, and distal cam follower 390d includes a plurality of distal cam followers 390d circumferentially spaced apart from one another around the valve shaft 320. The cam surface 380 includes a plurality of circumferentially spaced apart cam surfaces 380, each having a proximally-facing sloped surface 382p (facing the proximal cam followers 390p), and a distally-facing sloped surface 382d (facing the distal cam followers 390d). The proximal cam followers 390p have sloped surfaces 392p facing the proximally-facing sloped surface 382p of the cam surface 380, and the distal cam followers 390d have sloped surfaces 392d facing the distally-facing sloped surface 382d of the cam surface 380. The various sloped surfaces 382 and 392 extend transverse to a plane perpendicular to the actuation axis A. As may be appreciated with reference to FIGS. 6A-6E, distal axial movement of the actuatable member 310 axially moves the sloped surfaces 392p of the proximal cam followers 390p into engagement with the proximally-facing sloped surface 382p of the cam surface 380. Continued distal axial movement of the actuatable member 310 causes the sloped surfaces 392p of the proximal cam followers 390p to ride along the proximally-facing sloped surface 382p of the cam surface 380 and to cause rotation of the valve shaft 320 with respect to the collar 340 and thus with respect to the valve well 350. Similarly, proximal axial movement of the actuatable member 310 axially moves the sloped surfaces 392d of the distal cam followers 390d into engagement with the distally-facing sloped surface 382d of the cam surface 380. Continued proximal axial movement of the actuatable member 310 causes the sloped surfaces 392d of the distal cam followers 390d to ride along the distally-facing sloped surface 382d of the cam surface 380 and to cause further rotation of the valve shaft 320 with respect to the collar 340 and thus with respect to the valve well 350. As may be appreciated, rotation of the valve shaft 320 with respect to the valve well 350 shifts the transversely-extending valve shaft port 322 with respect to the transversely-extending valve well port 352 and into and out of fluid communication therewith.

Operation of an actuatable member 310 and actuation mechanism 370 such as illustrated in FIG. 5 may be better appreciated with reference to the sequential positions of the cam follower 390 with respect to the cam surface 380 illustrated in FIGS. 6A-6E (with arrows in FIG. 3A-3E indicating the next movement of the cam follower 290). The valve assembly 300 is illustrated in an off configuration in FIG. 6A, with the valve shaft 320 in an off position with the transversely-extending valve shaft port 322 not aligned with and not fluidly communicating with the transversely-extending valve well port 352. Moreover, the actuatable member 310 is in a neutral configuration in FIG. 6A, with no actuation force being applied thereto.

When an actuation force F is applied to the user-engagement element 330, the actuatable member 310 is moved distally toward the distal end 301 of the valve assembly 300, and the actuation mechanism 370 is activated, as illustrated in FIG. 6B. More particularly, the proximal cam followers 390p move distally with the user-engagement element 330, and the sloped surfaces 392p of the proximal cam followers 390p are brought into engagement with the proximally-facing sloped surface 382p of the cam surface 380. Since the collar 340 is fixed against rotation with respect to the valve well 350, continued axially distal engagement of the sloped surfaces 392p of the proximal cam followers 390p with the proximally-facing sloped surface 382p of the cam surface 380 causes the proximal cam followers 390p to ride along the proximally-facing sloped surface 382p of the cam surface 380 to cause rotation of the valve shaft 320 with respect to the valve well 350 (clockwise when viewed from the proximal end 313 of the actuatable member 310, or to the left in FIG. 6B, to the position in FIG. 6C). The transversely-extending valve shaft port 322 is thereby moved with respect to the transversely-extending valve well port 352, as illustrated in FIG. 6C. The cam surfaces 380 may define stop surfaces 386 which the proximal cam followers 390p meet during rotation of the valve shaft 320, and which prevent the proximal cam followers 390p from further rotation. As such, the stop surfaces 386 on the cam surfaces 380 may serve as distal limit stops for rotational (and typically also distal axial) movement of the valve shaft 320.

Removal of the actuation force F from the user-engagement element 330 allows the biasing element 312 to move the user-engagement element 330 proximally, toward the proximal end 303 of the valve assembly 300, as illustrated in FIG. 6C. Proximal movement of the valve shaft 320 brings the sloped surfaces 392d of the distal cam followers 390d into engagement with the distally-facing sloped surface 382d of the cam surface 380. Since the collar 340 is fixed with respect to the valve well 350 and therefore does not rotate, continued axially proximal engagement of the sloped surfaces 392d of the distal cam followers 390d with the distally-facing sloped surface 382d of the cam surface 380 causes the distal cam followers 390d to ride along the distally-facing sloped surface 382d of the cam surface 380 to cause further rotation of the valve shaft 320 (in the same direction caused by the sloped surfaces 392p of the proximal cam followers 390p riding along the proximally-facing sloped surface 382p of the cam surface 380). The transversely-extending valve shaft port 322 is thereby further moved with respect to the transversely-extending valve well port 352. In the example of an embodiment illustrated in FIG. 6E, the transversely-extending valve shaft port 322 is in fluid communication with the transversely-extending valve well port 352 when the actuatable member 310 is once again in a neutral position. It will be appreciated that the distal cam followers 390d may be stopped from further rotational movement by engaging the stop surfaces 386 on the cam surfaces 380. As such, the stop surfaces 386 on the cam surfaces 380 may serve as limit stops for rotational (and typically also proximal axial) movement of the valve shaft 320.

As may be appreciated with reference to FIGS. 6A-6E, and the above description thereof, the rotation of the valve shaft 220 caused by the distal movement of the valve shaft 220 (upon application of an actuation force F thereto), and the sloped surfaces 392p of the proximal cam followers 390p riding along the proximally-facing sloped surface 382p of the cam surface 380, causes the valve shaft 220 to rotate 45° from a first neutral position. And, the further rotation of the valve shaft 320 caused by the proximal movement of the valve shaft 320, and the sloped surfaces 392d of the distal cam followers 390d riding along the distally-facing sloped surface 382d of the cam surface 380, causes the valve shaft 320 to rotate 45° further from the first neutral position. As such, the valve shaft 320 is rotated 90° at the completion of actuating movement thereof (application and release of an actuation force F to the actuatable member 310) to move the transversely-extending valve shaft port 322 into or out of alignment with the transversely-extending valve well port 352. The down and up motion is repeated to rotate the valve shaft 90 degrees to move the transverse hole through the valve shaft back into and out of alignment and fluid communication with the transversely-extending port in the valve well.

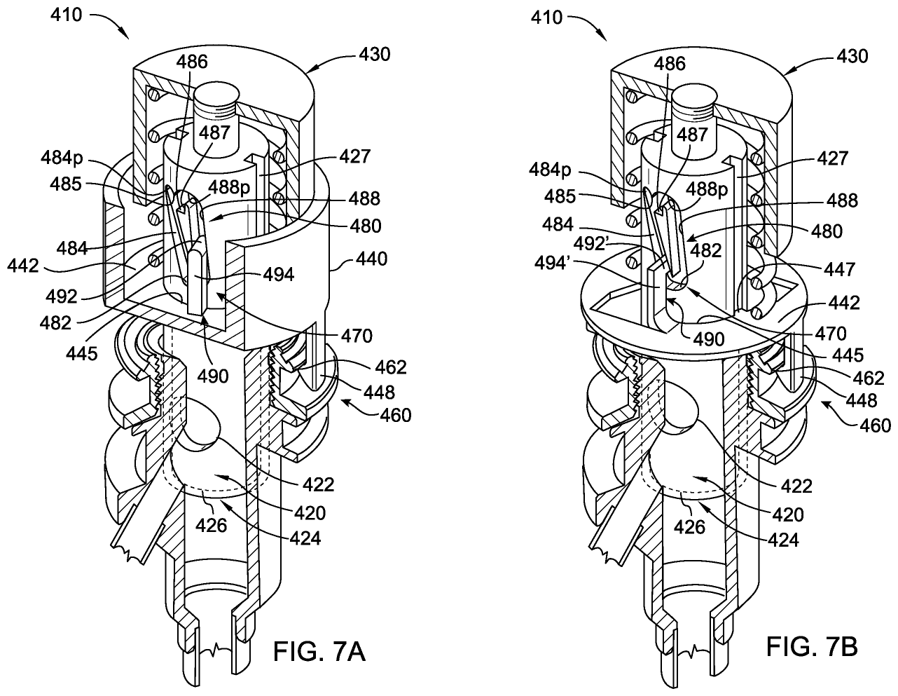

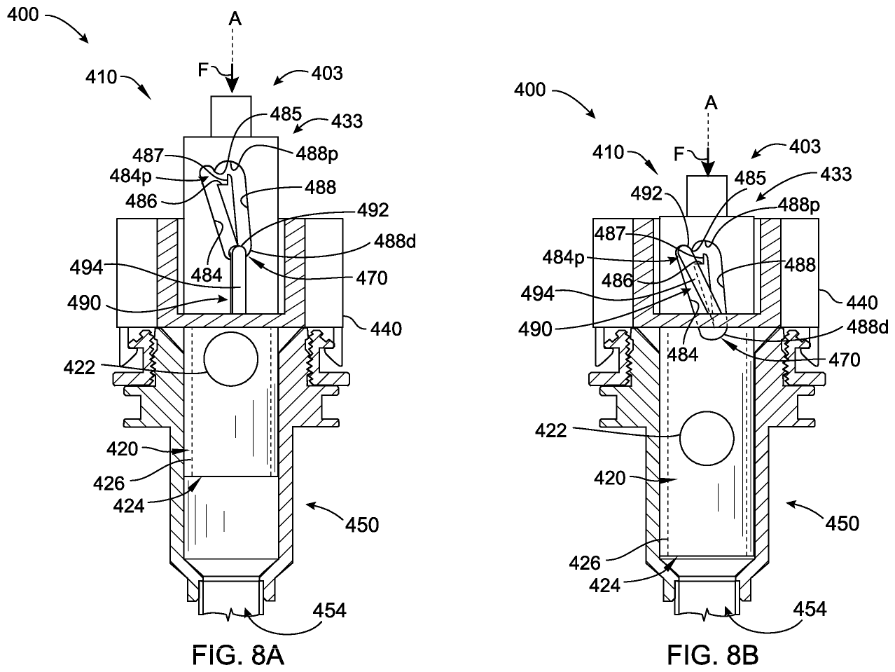

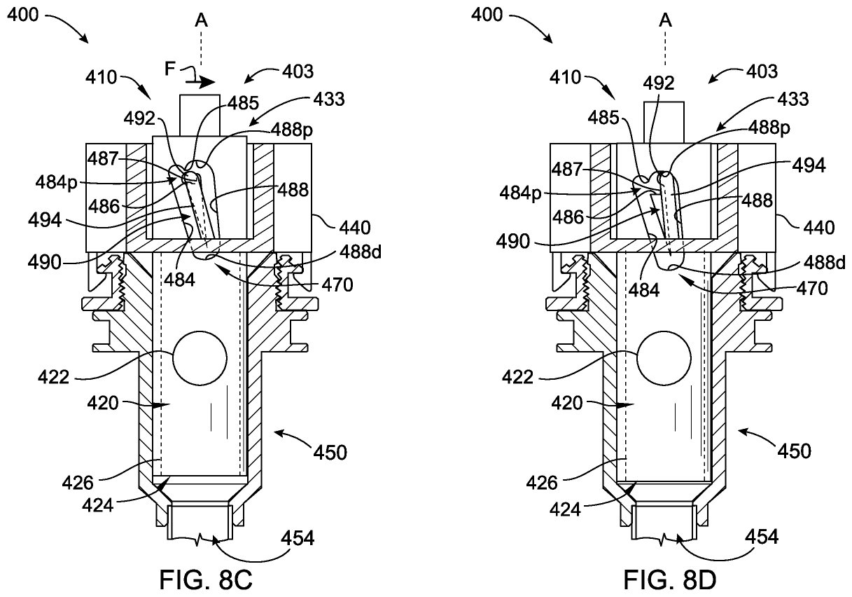

As noted above, principles of the present disclosure encompass various arrangements and configurations of actuation mechanisms arranged and configured with respect to components of a valve assembly to shift the valve assembly between stable on and off configurations, with an actuatable member remaining in a neutral configuration in either configuration of the valve assembly. For instance, instead of a cam follower being associated with a valve shaft of the actuatable member, and a cam surface being associated with a collar of the valve assembly, such as in the above-described example of an embodiment of an actuation mechanism illustrated in FIG. 5 and FIGS. 6A-6D, as noted above, a generally reverse configuration is within the scope and spirit of the present disclosure. An example of an embodiment of a valve assembly with an actuation mechanism with a cam follower associated with a collar of the valve assembly, and a cam surface associated with a portion of the actuatable member of the valve assembly is illustrated in FIG. 7 and FIGS. 8A-8D. For the sake of convenience, and without intent to limit, components illustrated in FIG. 7 and FIGS. 8A-8D which are similar to components illustrated in FIG. 5, FIGS. 6A-6E are indicated with similar reference numbers increased by 100 and reference is made to the descriptions thereof in connection with FIGS. 3A-3E for the sake of brevity and without intent to limit. As in the above-described examples of embodiments, the actuation mechanism is positioned and configured to engage with an actuatable member and a collar of a valve assembly to cause shifting of a transversely-extending port in a valve shaft of the actuatable member with respect to a transversely-extending port in a valve well of the valve assembly to shift the valve assembly between on and off configurations, with the actuatable member remaining in a neutral configuration in either configuration of the valve assembly. However, in contrast with the above-described examples of embodiments, the transversely-extending port in the valve shaft is moved axially in and out of fluid communication with the transversely-extending port in the valve well (in contrast with the rotational movement described with respect to the above-described examples of embodiments).

In the example of an embodiment of an actuatable member 410 illustrated in isolation in FIG. 7A, FIG. 7B, and illustrated mounted with respect to a valve assembly 400 in FIGS. 8A-8D, an actuation mechanism 470 includes a cam surface 480 and a cam follower 490. Like the example of an embodiment illustrated in FIG. 5 and FIGS. 6A-6E, a collar 440 is positioned around the actuatable member 410, and the actuation mechanism 470 is positioned with respect to the actuatable member 410 and the collar 440. The collar 440 is mounted with respect to a valve well 450 of a valve assembly 400 to be rotationally fixed with respect to the valve well 450, such as in a manner known to those of ordinary skill in the art. In the example of an embodiment illustrated in FIG. 7A, FIG. 7B, and FIGS. 8A-8D, the collar 440 is mounted with respect to the valve well 450 in a manner such as illustrated in FIG. 2, FIGS. 3A-3E, and FIG. 4. Accordingly, components illustrated in FIG. 7A, FIG. 7B, and FIGS. 8A-8D which are similar to components illustrated in FIG. 2, FIGS. 3A-3E, and FIG. 4 are indicated with similar reference numbers increased by 200 and reference is made to the descriptions thereof in connection with FIG. 2, FIGS. 3A-3E, and FIG. 4 for the sake of brevity and without intent to limit.

In contrast with the example of an embodiment illustrated in FIG. 5 and FIGS. 6A-6E, in the example of an embodiment of an actuation mechanism 470 mounted with respect to and actuatable member 410 illustrated in FIG. 7A, FIG. 7B, and FIGS. 8A-8E, the cam surface 480 is mounted on an outer surface of the valve shaft 420 of the actuatable member 410, and the cam follower 490 extends from a collar 440 positioned around the actuatable member 410 to interact with the cam surface 480. Also in contrast with the example of an embodiment illustrated in FIG. 5 and FIGS. 6A-6E, instead of the valve shaft 420 rotating between on and off positions thereof, the valve shaft 420 of the example of an embodiment illustrated in FIG. 7A, FIG. 7B, and FIGS. 8A-8E axially moves, along the actuation axis A, between on and off positions thereof. More particularly, the valve shaft 420 extends through a shaft-retaining opening 445 in a shaft retainer wall 442 extending radially inwardly from a skirt 444 of the collar 440. The valve shaft 420 has an axially-extending groove 427 engaged by a radially-inwardly extending projection 447 on the collar 440 (e.g., radially-inwardly extending from the shaft retainer wall 442). The valve shaft 420 is thereby rotationally held with respect to the collar 440 to not rotate with respect thereto. As the collar 440 is mounted with respect to the valve well 450 in a manner as described above with respect to the example of an embodiment of FIG. 2, FIGS. 3A-3E, and FIG. 4, the collar 440 does not rotate with respect to the valve well 450, and the valve shaft 420 (which is rotationally fixed with respect to the collar 440) thus also does not rotate with respect to the valve well 450. As such, axial translation of the actuatable member 410 along the actuation axis A, such as upon application of an actuation force F to the user-engagement element 430 thereof, causes axial translation of the valve shaft 420 to move the transversely-extending port 422 defined therethrough with respect to the transversely-extending port 452 defined in the valve well 450.

The cam surface 480 of the actuation mechanism 470 provides an off stop surface 482 for the cam follower 490 to hold the valve shaft 420 in an off position with respect to the valve well 450. When the actuatable member 410 is moved distally along the actuation axis A from an off position to an on position, the cam follower 490 rides along an on sloped surface 484 from the off stop surface 482 to an on stop surface 486. When the cam follower 490 is seated in the off stop surface 482, the valve shaft 420 is held in a stable on position with respect to the valve well 450. The valve shaft 420 may remain in such stable on position even when no actuation force F is applied thereto, until a further actuation force F is applied to move the actuatable member 410 back to an off position. When the actuatable member 410 is moved distally along the actuation axis A from an on position to an off position, the cam follower 490 rides along and an off sloped surface 488 from the on stop surface 486 back to off stop surface 482 to shift the valve shaft 420 from a stable on position to a stable off position. Optionally, a biasing element 412 biases the user-engagement element 430 proximally upon removal of the actuation force F applied thereto to shift the position of the valve shaft 420 so that the valve shaft 420 moves to a stable on or off position upon removal of an actuation force F applied to the user-engagement element 430.

In the example of an embodiment of an actuation mechanism 470 illustrated in FIG. 7A and in FIG. 7B, the cam follower 490 includes a radially-inwardly extending cam finger 492 configured to engage, sequentially, the off stop surface 482, the on sloped surface 484, the on stop surface 486, and the off sloped surface 488 of the cam surface 480 as the valve shaft 420 is shifted axially along the actuation axis A and between off and on positions to shift the valve assembly 400 between off and on configurations. The cam finger 492 may be laterally biased in a direction transverse to the actuation axis A, such as to facilitate movement of the cam finger 492 with respect to the various features of the cam surface 480. In the example of an embodiment illustrated in FIG. 7A, the cam finger 492 is mounted on a cam-biasing element 494 mounted with respect to the shaft retainer wall 442 to be biased to a generally vertical neutral position (e.g., along the actuation axis A). In the example of an embodiment illustrated in FIG. 7B, the cam finger 492 is mounted on a cam-biasing element 494′ which is stamped from a separate wall 442′, which may be positioned with respect to the shafter retainer wall 442 so that the cam finger 492 may extend into the cam surface 480. For the sake of convenience, and without intent to limit, references to a cam-biasing element 494 herein are intended to encompass references to the cam-biasing element 494′ illustrated in FIG. 7B. In some embodiments, the cam-biasing element 494 is biased out of its neutral position when the cam follower 490 is positioned with respect to at least one of the stop surfaces 482, 486 and/or with respect to at least one of the sloped surface 484, 488. For instance, the cam follower 490 may be biased out of its neutral position when resting in at least one of the stop surfaces 482, 486 to bias the cam finger 492 into position with respect to the associated sloped surface 484, 488 to cause the cam finger 492 to move to the other of the stop surfaces 482, 486.

In the example of an embodiment of an actuation mechanism 470 illustrated in FIG. 7A, FIG. 7B, and FIGS. 8A-8E, the distal position of the valve shaft 420, closer to the distal end 401 of the valve well 450, is the on position, and the proximal position of the valve shaft 420, closer to the proximal end 403 of the valve well 450, is the off position. However, the present disclosure encompasses a reverse configuration with appropriate modifications which may be made by those of ordinary skill in the art. Operation of an actuation mechanism 470 formed in accordance with various principles of the present disclosure may be better appreciated with reference to the sequential positions of the cam follower 490 with respect to the cam surface 480 illustrated FIGS. 8A-8D.

Operation of an actuation mechanism 470 such as illustrated in FIG. 7A and FIG. 7B may be appreciated with reference to the sequential positions of the cam follower 209 with respect to the cam surface 230 illustrated in FIGS. 8A-8C. It will be appreciated that the user-engagement element 430 of the actuatable member 410 has been left out in order to simplify illustration, and not to represent that the actuatable member 410 does not have a user-engagement element 430. The valve assembly 400 is illustrated in an off configuration in FIG. 8A, with the valve shaft 420 in an off position with the transversely-extending valve shaft port 422 not aligned with and not fluidly communicating with the transversely-extending valve well port 452. The cam finger 492 is positioned in the off stop surface 482 at a distal end 488d of the off sloped surface 488. As may be appreciated, such position may prevent further proximal movement of the valve shaft 420 (e.g., as biased towards the proximal end 403 of the valve assembly 400 by the biasing element 412), and thus may serve as a limit stop limiting proximal movement of the valve shaft 420 with respect to the valve well 450.

To shift the valve assembly 400 into an on configuration, the actuatable member 410 is moved distally, such as illustrated in FIG. 8B. For instance, application of a distally directed actuation force F to the user-engagement element 430 at the proximal end 413 of the actuatable member 410 moves the actuatable member 410 distally from the proximal end 403 of the valve assembly 400 toward the distal end 401 of the valve assembly 400. The cam finger 492 is positioned with respect to the on sloped surface 484 of the cam surface 480 such that the cam finger 492 will ride upwardly along the on sloped surface 484 as distal movement of the actuatable member 410 moves the valve shaft 420 and thus the cam surface 480 distally. The proximal end 484p of the on sloped surface 484 may function as a limit stop for distal movement of the actuatable member 410 (towards the distal end 401 of the valve assembly 400) since, upon the cam finger 492 reaching the proximal end 484p of the on sloped surface 484, the cam finger 492 cannot move further proximally, and the valve shaft 420 thus cannot move further distally. It will be appreciated that when the cam finger 492 is positioned at the proximal end 484p of the on sloped surface 484, the cam-biasing element 494 is biased laterally away from its neutral position (e.g., biased away from the actuation axis A along which the cam-biasing element 494 extends in a generally vertical neutral position) and the biasing element 412 of the actuatable member 410 is compressed. A lateral stop 485 may be provided with respect to the proximal end 484p of the on sloped surface 484 to stop the cam-biasing element 494 from returning to its neutral position.

Removal of the actuation force F from the user-engagement element 430 allows the biasing element 412 to move the actuatable member 410 proximally, toward the proximal end 403 of the valve assembly 400, as illustrated in FIG. 8C. Such proximal movement of the actuatable member 410 causes proximal movement of the valve shaft 420 to move the lateral stop 485 of the cam surface 480 proximally as well, to allow the cam-biasing element 494 to move laterally towards its neutral position (e.g., towards the actuation axis A). However, as the cam finger 492 moves laterally towards its neutral position, and the valve shaft 420 moves proximally, the cam finger 492 engages the on stop surface 486. Further proximal movement of the valve shaft 420 is thereby inhibited, holding the valve shaft 420 in an on configuration, as illustrated in FIG. 8C. A lateral stop 487 may be provided along the on stop surface 486 to inhibit further lateral movement of the cam-biasing element 494 towards its neutral position, further stabilizing the position of the cam finger 492 with respect to the on stop surface 486. The valve shaft 420 is thereby stably held in an on position with the valve shaft transversely-extending port 422 in fluid communication with the valve well transversely-extending port 452, as illustrated in FIG. 8C.

To return the valve shaft 420 to an off position from an on position, a further actuation force F is applied to the actuatable member 410 (e.g., to the user-engagement element 430) in a distal direction (towards the distal end 401 of the valve assembly 400), as illustrated in FIG. 8D. Such actuation force F moves the valve shaft 420 distally with respect to the cam finger 492 so that the lateral stop 487 along the on stop surface 486 is moved distal to the cam finger 492 and the cam-biasing element 494 may continue to bias the cam finger 492 to its neutral position. The distal movement of the valve shaft 420 with respect to the cam finger 492 also moves the cam finger 492 into engagement with the proximal end 488p of the off sloped surface 488. The proximal end 488p of the off sloped surface 488 may function as a limit stop for distal movement of the actuatable member 410 (towards the distal end 401 of the valve assembly 400) along the off sloped surface 488 since, upon the cam finger 492 reaching the proximal end 488p of the off sloped surface 488, the cam finger 492 cannot move further proximally, and the valve shaft 420 thus cannot move further distally. It will be appreciated that when the actuatable member 410 is in the position illustrated in FIG. 8B as well as in the position illustrated in FIG. 8C, the biasing element 412 is compressed and may serve as a further limit stop to distal movement of the actuatable member 410 with respect to the valve well 450

Removal of the actuation force F from the user-engagement element 430 when the cam finger 492 is positioned at the proximal end 488p of the off sloped surface 488 allows the biasing element 412 to move the actuatable member 410 proximally, toward the proximal end 403 of the valve assembly 400. The proximal end 488p of the off sloped surface 488 may be sloped to move the cam finger 492 laterally, and/or the cam-biasing element 494 may remain biased to move the cam finger 492 laterally, to the other side of the lateral stop 487 and toward the off sloped surface 488. Thus, continued movement proximal movement of the valve shaft 420 (such as caused by the biasing element 412) allows the cam finger 492 to ride along the off sloped surface 488 and back to the off stop surface 482. Once the cam finger 492 reaches the off stop surface 482, the actuatable member 410 cannot move further proximally, and the valve shaft 420 remains in the off position, with the valve shaft transversely-extending port 422 not in fluid communication with the valve well transversely-extending port 452, until an actuation force F is applied to the actuatable member 410 again. The actuatable member 410 once again is in a neutral position, such as illustrated in FIG. 8A, with the valve shaft 420 in an off position and the valve assembly 400 in an off configuration.

As noted above, in the example of an embodiment of an actuatable member 210 and associated actuation mechanism 270 illustrated in FIG. 2, FIGS. 3A-3E, and FIG. 4, and the example of an embodiment of an actuatable member 310 and associated actuation mechanism 370 illustrated in FIG. 5, FIGS. 6A-6D, the associated valve shaft 220, 320 rotates between on and off positions. However, the axial position of the valve shafts 220, 320 with respect to the associated valve well 250, 350 may be in the same neutral position in both the on position as well as the off position. In accordance with various principles of the present disclosure, an indicator such as a window may be provided along the actuatable member 210, 310 to indicate the position of the valve shaft 220, 320 and/or the configuration of the associated valve assembly 200, 300.—For instance, the position of an indicator on the valve shaft 220, 320 relative to the-engagement element 230, 330 may indicate the position of the valve shaft 220, 320 relative to the user-engagement element 230, and thus the collar 240 and the valve well 250 and the valve well transversely-extending port 252.

Various further benefits of the various aspects, features, components, and structures of a valve shaft and associated seal members, as well as valve assemblies and endoscopes such as described above, in addition to those discussed above, may be appreciated by those of ordinary skill in the art.

It is to be understood by one of ordinary skill in the art that the present discussion is a description of illustrative examples of embodiments only, and is not intended as limiting the broader aspects of the present disclosure. It will be appreciated that principles of the present disclosure may be applied to various medical devices, instruments, tools, etc., such, without limitation, a variety of medical devices, instruments, tools, etc., for accessing anatomical sites and applying suction and/or irrigation thereto, including, for example, endoscopes, gastroscopes, duodenoscopes, catheters, ureteroscopes, bronchoscopes, colonoscopes, arthroscopes, cystoscopes, hysteroscopes, and the like, having integrated features for suction and/or irrigation of anatomical sites. Moreover, principles of the present disclosure may be applied to reusable or single-use devices, instruments, tools, etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. For instance, various elements and components of a valve assembly described herein may be coupled or engaged directly or indirectly with one another, regardless of how such connections are depicted in the drawings. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. An actuatable member for a valve assembly of a medical instrument, said actuatable member having a proximal end and a distal end and comprising:
a user-engagement element along the proximal end thereof; and
a shaft along the distal end thereof;
wherein:
said shaft is positionable within a valve well of the valve assembly and shiftable in the valve well along an actuation axis and between an on position in which the valve assembly is in an on configuration, and an off position in which the valve assembly is in an off configuration; and
said actuatable member is held in each of the on position and the off position without an actuation force being applied thereto.

2. The actuatable member of claim 1, wherein said actuatable member is held in each of the on position and the off position by an actuation component on one of said shaft or said user-engagement element.

3. The actuatable member of claim 1, wherein said user-engagement element is movable with respect to said shaft.

4. The actuatable member of claim 3, wherein said user-engagement element and said shaft rotate with respect to each other to shift said shaft between the on position and the off position.

5. The actuatable member of claim 4, wherein said user-engagement element moves axially along said actuation axis and with respect to said shaft.

6. The actuatable member of claim 4, wherein said user-engagement element and said shaft together move axially along said actuation axis.

7. The actuatable member of claim 1, wherein said shaft includes one of a cam surface or a cam follower configured to effect movement of said shaft between the on position and the off position upon engagement with the other of a cam surface or a cam follower associated with the valve assembly.

8. The actuatable member of claim 7, wherein said shaft includes a proximal cam surface and a distal cam surface each extending circumferentially around said shaft.

9. The actuatable member of claim 8, wherein said user-engagement element includes a radially-inwardly directed cam follower engaging said cam surfaces of said shaft to rotate said shaft between the on position and the off position.

10. The actuatable member of claim 9, wherein said cam follower holds said cam surfaces alternately in the on position or the off position.

11. The actuatable member of claim 7, wherein said shaft includes one of a movable cam follower or a vertically-extending cam surface having an on rest position for the cam follower in which the cam follower holds said shaft in the on position, and an off position for the cam follower in which the cam follower holds said shaft in the off position.

12. The actuatable member of claim 1, wherein said shaft rotates between the on position and the off position.

13. The actuatable member of claim 1, wherein said shaft axially shifts between the on position and the off position.

14. The actuatable member of claim 1, further comprising a biasing element positioned to bias said user-engagement element proximally to a neutral position, the neutral position alternating between the on position and the off position upon sequential application and removal of a distal actuation force to said user-engagement element.

15. An actuatable member assembly for a valve assembly of a medical instrument, said actuatable member assembly comprising:
an actuatable member comprising a user-engagement element along a proximal end thereof, and a shaft along a distal end thereof; and
a collar extending circumferentially around said shaft and configured to be operatively engaged with the valve assembly to mount said actuatable member with respect to the valve assembly;
wherein:
said shaft is positionable within a valve well of the valve assembly and shiftable in the valve well along an actuation axis and between an on position in which the valve assembly is in an on configuration, and an off position in which the valve assembly is in an off configuration; and
one of said user-engagement element, said shaft, or said collar includes a cam surface, and another of said user-engagement element, said shaft, or said collar includes a cam follower configured to engage said cam surface to effect movement of said shaft between the on position and the off position.

16. The actuatable member assembly of claim 15, further comprising a biasing element positioned to bias said user-engagement element proximally to a neutral position, the neutral position alternating between the on position and the off position upon sequential application and removal of a distal actuation force to said user-engagement element to move said cam follower along said cam surface.

17. A method of actuating a valve assembly of a medical device, said method comprising:
applying an actuation force to an actuatable member of the valve assembly and releasing the actuation force, leaving the valve assembly in one of an on configuration or an off configuration; and
applying an additional actuation force to the actuatable member and releasing the actuation force, leaving the valve assembly in the other of an on configuration or an off configuration.

18.

19. The method of claim 17, wherein the actuatable member is in a neutral position without application of an actuation force thereto, and a biasing element biases the actuatable member back to the neutral position upon release of an actuation force.

20. The method of claim 18, wherein application of an actuation force to the actuatable member commences shifting of the valve assembly from one of an on configuration or an off configuration to the other of an on configuration or an off configuration, and release of the actuation force allows the biasing element to complete shifting of the valve assembly from one of an on configuration or an off configuration to the other of an on configuration or an off configuration so that the valve assembly remains in the other of an on configuration or an off configuration when the actuatable member returns to its neutral position.

21. The method of claim 17, wherein releasing the actuation force allows the valve assembly to shift from one of an on configuration or an off configuration to the other of an on configuration or an off configuration.
